(19) United States
(12) Patent Application Publication
(10) Pub. No.: US 2008/0050001 A1
(43) Pub. Date: Feb. 28, 2008

(54) Use of Subsets of the Acquired Data to Improve the Diagnostic Outcome in Cardiac SPECT Imaging

(75) Inventor: Richard Conwell, Del Mar, CA (US)

Correspondence Address: Law Office of Scott C Harris, PO Box 1389, Rancho Santa Fe, CA, 92067, US

(73) Assignee: DIGIRAD CORPORATION

(21) Appl. No.: 11/846,445

(22) Filed: Aug. 28, 2007

Related U.S. Application Data
(60) Provisional application No. 60/840,897, filed on Aug. 28, 2006.

(51) Int. Cl. G06K 9/60; A61B 5/02

(52) U.S. Cl. 382/131; 382/128; 600/508

(57) ABSTRACT

Undesired effect reduction in a medical image. A set of medical images corresponding to a number of images each taken at a different time and from plural different projection views are obtained. The images are analyzed to determine which images in the set have effects from an undesired effect, such as patient movement or attenuation due to an organ. The undesired images are removed to form a subset which may be a subset over a specified angle range, or just certain images within the subset. The medical analysis is carried out on the subset of images.

### CROSS-REFERENCE TO RELATED APPLICATIONS

This application claims priority from Provisional Application No. 60/840,897, filed Aug. 28, 2007, the contents of which are herewith incorporated by reference.

### BACKGROUND

SPECT imaging takes images of body organs and parts that have uptaken radiopharmaceutical agents. Cardiac SPECT is a special kind of imaging where the uptake is by the cardiac wall. However, the amount of cardiac wall uptake is often different from the uptake of surrounding tissues, such as lung tissues. This allows for reconstruction of a 3D-image of the patient's heart using ECT (emission computed tomography) approaches.

Physicians/cardiologists can extract physiological information from the reconstructed images such as perfusion abnormalities, ejection fraction, and others. The information thus obtained can be used for diagnosis.

The diagnostic outcome of cardiac SPECT imaging may be adversely affected by certain physical phenomena.

For example, breast attenuation may introduce inferior wall artifacts when patients are imaged in the upright position, or anterior wall artifacts when patients are imaged in the supine position. The diagnostic outcome can be improved if the artifacts introduced by breast attenuation can be mitigated.

Patient motion can introduce a variety of artifacts into the reconstructed images. Diagnostic outcome can be improved if motion introduced artifacts can be reduced or eliminated.

Tissue attenuation in cardiac SPECT imaging has been addressed by a number of techniques in the prior art.

Attenuation correction in image reconstruction has been suggested. This approach requires generating an attenuation map and then modeling attenuation correction in image reconstruction algorithms. The attenuation map generation may require additional hardware/software (such as a transmission device and transmission reconstruction). The modeling is relatively complex, due to the scatter effect.

Another technique relies on the physician reading the raw data in a cine display to identify if breast attenuation or diaphragmatic attenuation exists.

Sequential prone and supine images may be obtained to change the distribution of the attenuating tissues around the heart. This approach is complex, because it requires two sequential scans. Also, this approach might not provide the desired results when the patient is imaged in the upright position.

Patient motion in cardiac SPECT imaging can also be addressed, for example by performing motion correction on the acquired data. The accuracy of this approach depends on the algorithm used, but all motion correction relies on large amounts of data. The accuracy decreases when the count density is very low. The accuracy also decreases when there is overlap of the heart and liver or other organs with high uptakes.

Motion correction in image reconstruction can be carried out by recording the motion using motion tracking devices during patient scans. This technique is limited by the accuracy of the motion tracking system as well as the cost and space requirement of the systems.

Mapping of motion-introduced artifacts with the motion observed in the raw data in cine display can be done also. This approach is not accurate when patient motion is small.

At least one reported work uses a subset of the acquired data for image comparison, such as 360° versus 180° comparison. As far as we know, none of those were used to address tissue attenuation or patient motion for the improvement of diagnostic outcome of cardiac SPECT imaging.

### SUMMARY

Diagnostic outcome of cardiac SPECT imaging by identifying tissue attenuation artifacts and reducing/identifying motion artifacts by selecting subsets of all the images collectively representing different projection views.

### BRIEF DESCRIPTION OF THE DRAWINGS

FIG. 1 shows the data acquisition over 202° from RAO 45° to LPO 67° and a subset of the data in 180° from RAO 23° to LPO 67°;

FIG. 2 shows a subset of data with coughing occurring in the second projection view;

FIG. 3 shows a subset of data that excludes the projection views that include patient motion, e.g. from the patient coughing: and

FIG. 4 is a flowchart showing an embodiment.

### DETAILED DESCRIPTION

This disclosure describes a unique approach to address issues such as those stated above to improve the diagnostic outcome of cardiac SPECT imaging.

In this approach, one or more subsets of the full set of acquired data (“the full data”) are generated. Multiple images are reconstructed from the subsets of the full data, as well as from the full data itself.

Subsets are generated from the full data so that they satisfy the data completeness condition. The data in the subsets have different degrees of physical effects that cause imperfect medical imaging, e.g., those described above. Therefore, the images reconstructed from the subsets may provide complemental information to the image reconstructed from the full data. Note that this disclosure also includes the techniques to acquire the full data that satisfy the requirement to generate the above data subsets.

An embodiment operates to improve the diagnostic outcome when breast attenuation presents. In that case, one can acquire the full data from RAO 45° to LPO 67° in a range of 202°. The full data allows one to generate one data subset from RAO 23° to LPO 67° in a range of 180°. The subsets will have much less attenuation from the right breast as compared with the full data, thus the image reconstructed from such a subset will show less breast attenuation, and will thus provide complemental information to the image reconstructed from the full data. This may improve the diagnostic outcome.

In another example, when the patient undergoes a large amount of motion, but the motion lasts for a very short period of time during the acquisition, (such as during coughing), such motion can very likely be significant only in one or a few projection views. The full data allows generating one data subset that does not include the projection view that has the large motion. The image reconstructed from this subset will be essentially motion free. This assists the physician in assessing the motion effect when reading the image reconstructed from the full data for diagnosis, and thus improves the diagnostic outcome.

The techniques described in this application include advantages. It only requires one scan. It allows for the assessment of the effects of tissue attenuation as complementary information for the improvement of diagnostic outcome. It also allows for the assessment of the effects of certain types of patient motion (such as coughing) for the improvement of diagnostic outcome.

The techniques can be used for any computed tomography when the reconstruction of a subset of the acquired data can provide complemental information to improve diagnostic outcome. The subset of the data is generated from the full data so that it includes different degrees of the physical effects than the full data. Therefore, the reconstructed image from the subset may show different degrees of the physical effects than the image reconstructed from the full data.

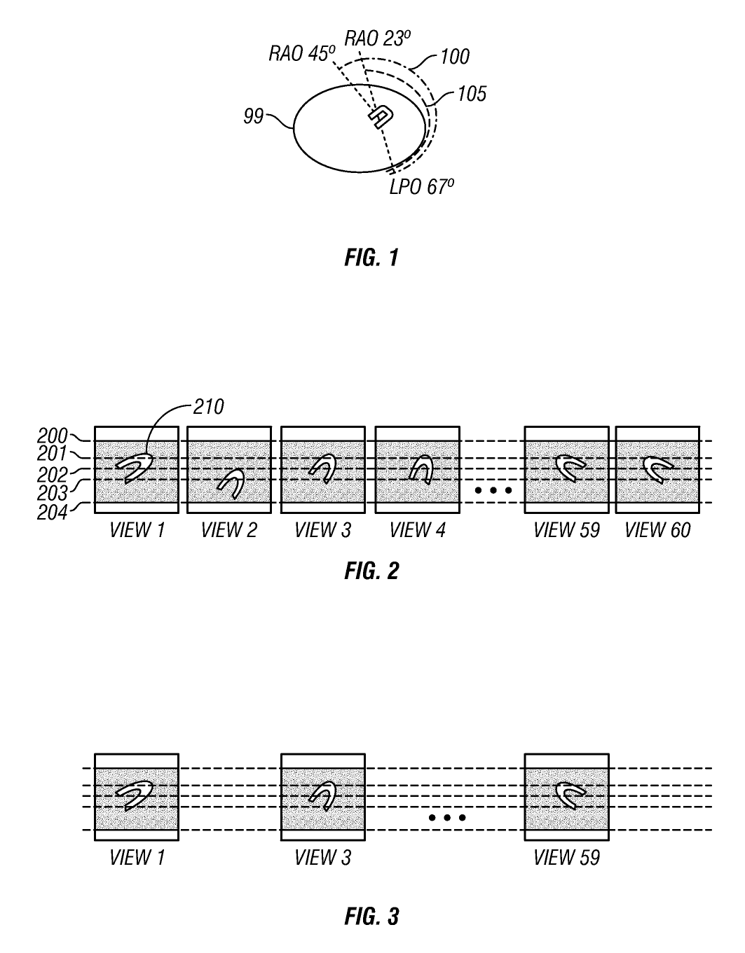

FIG. 1 illustrates the angular setup of the full data and a subset of the data to address the breast attenuation effect. Data of an imaged body 99 is acquired over the 202 degree area 100, from RAO 23 to LPO 67. A subset 105 of the data is over 180 degrees from RAO 23 to LPO 67. The subset 105 of the data is from an area that is less attenuated by right breast attenuation than the full data. Hence a reconstructed image from the subset of the data 105 will show less breast attenuation than the image from the full data 100.

FIGS. 2 and 3 demonstrate the use of subsets of the full data to exclude projection views that have large patient motion.

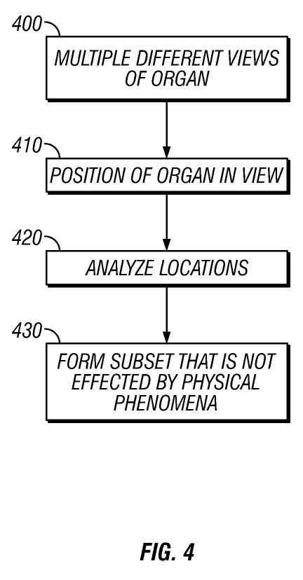

The embodiment is processed according to the flowchart of FIG. 4. At 400, multiple different views representing a view of the imaged body 99 are obtained. Each subset image is analyzed. Location lines 200, 201, 202, 203, 204 are used to determine the position of the organ 210, within the frame of the image at 410. View 1, for example, shows the edge of the organ against line 201. However, in view 2, the organ 210, is against line 202.

A first analysis at 420 determines if a subset image or an image reconstructed from the subset has reduced motion effect compared with the image reconstructed from the full data. For example, the analysis shows that view 2 has the organ in a different place than a majority of the other views. A subset of the views, shown in FIG. 3, excludes those views (here, e.g., view 2) that have the organ in a different place. Hence, the characteristics of a subset can assess whether an apparent defect in the images reconstructed from the full data is a true defect or is just an artifact introduced by patient motion, and the motion induced defect views 430 can thus be removed.

This embodiment may compensate for motion effects via generation of the data subsets from the original data so that each subset includes different degrees/patterns of the patient motion. Therefore, images reconstructed from the subsets are simplified for the assessment of motion effect, and in turn, can help the physicians to differentiate motion artifacts from perfusion defects, etc.

Another embodiment is used to improve the diagnostic outcome when breast attenuation presents. Two major steps are used for this embodiment. Data is acquired, e.g., as in FIG. 1, so that the subset of the data is complete, over an angular range greater or equal to 180° For this embodiment, the subset of the data may simply be according to a section of the scan (here sub scan 105) that is less effected by breast attenuation.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, while an embodiment describes cardiac “SPECT” imaging, this can be used for any kind of medical scan. Also, while this system describes only a few specific issues in imaging, such as movement and tissue attenuation, it can be used for a host of other applications.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation.

The computer that carries out the processing may be an Intel (e.g., Pentium or Core 2 duo) or AMD based computer, running Windows XP or Linux, or may be a Macintosh computer. The computer may also be a handheld computer, such as a PDA, cellphone, or laptop.

The programs may be written in C or Python, or Java, Brew or any other programming language. The programs may be resident on a storage medium, e.g., magnetic or optical, e.g. the computer hard drive, a removable disk or media such as a memory stick or SD media, wired or wireless network based or Bluetooth based Network Attached Storage (NAS), or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

What is claimed is

1. A method, comprising:
obtaining a full set of medical images of a body, from plural different projection views;
analyzing the set of medical images to determine at least one aspect of the images which was affected by an undesired effect during the obtaining of the set of images;
based on said analyzing, selecting a subset of the images which are less affected by said undesired effect; and
based on only said subset, and not on the full set, second analyzing at least a part of an aspect within said subset.

2. A method as in claim 1, further comprising carrying out a medical diagnosis based on the medical image.

3. A method as in claim 1, wherein said undesired effect is patient motion during the image obtaining which lasts for a time indicative of only part of the image obtaining.

4. A method as in claim 1, wherein said undesired effect is attenuation of a resulting image due to passage through tissue.

5. A method as in claim 4, wherein said undesired effect is attenuation due to passage through breast tissue.

6. A method as in claim 3, wherein said selecting a subset comprises selecting a subset of images attributable to a time when the patient is not in motion.

7. A method as in claim 4, wherein said selecting a subset comprises selecting a subset of images which has less effect due to the attenuation.

8. A method as in claim 1, wherein said subset of images is a subset of images which is less affected by said undesired effect.

9. A method as in claim 1, wherein said subset of images comprises individual images within the set.

10. A method as in claim 1, wherein said subset of images comprises a range of images within the set.

11. A method as in claim 1, wherein said obtaining a set of images comprises using cardiac SPECT images to obtain said image set.

12. A method as in claim 1, wherein said second analyzing comprises a tomographic reconstruction.
